(19) United States
(12) Patent Application Publication
(10) Pub. No.: US 2007/0275985 A1
(43) Pub. Date: Nov. 29, 2007

(54) Triazolopyrimidines

(75) Inventors: Olaf Gebauer, Koln (DE); Ulrich Heinemann, Leichlingen (DE); Hans-Ludwig Elbe, Wuppertal (DE); Herbert Gayer, Monheim (DE); Stefan Herrman, Langenfeld (DE); Jorg Nico Greul, Leichlingen (DE); Bernd-Wieland Kruger, Bergisch Gladbach (DE); Stefan Hillebrand, Neuss (DE); Ronald Ebbert, Nurnberg (DE); Ulrike Wachendorff-Neumann, Neuwied (DE); Peter Dahmen, Neuss (DE); Karl-Heinz Kuck, Langenfeld (DE)

Correspondence Address: STERNE, KESSLER, GOLDSTEIN & FOX P.L.L.C., 1100 NEW YORK AVENUE, N.W., WASHINGTON, DC, 20005, US

(73) Assignee: Bayer Cropscience Aktiengesellschaft, Monheim (DE)

(21) Appl. No.: 10/559,102

(22) PCT Filed: Jun. 1, 2004
(86) PCT No.: PCT/EP04/05876

§ 371 Date: Mar. 22, 2007

(30) Foreign Application Priority Data
Jun. 4, 2003 (DE) 10325133.2

(51) Int. Cl. A61K 31/519; A61P 31/04; C07D 211/70; C07D 239/24; C07D 487/04

(52) U.S. Cl. 514/262.1; 544/263; 544/334; 546/341

(57) ABSTRACT

The invention relates to new triazolopyrimidines of the formula 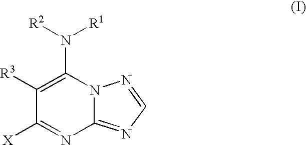 in which
R¹, R², R³ and X have the meanings specified in the description, a process for preparing these substances and their use for combating undesirable micro-organisms.
   The invention further relates to new intermediate products of the formulae 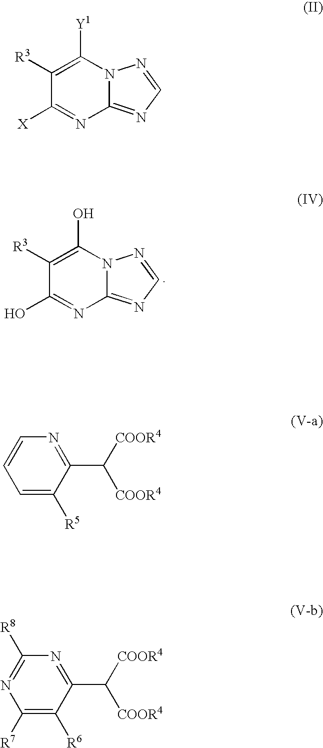 and processes for preparing substances.

The present invention relates to new triazolopyrimidines, a process for their preparation and their use for combating undesirable micro-organisms. The invention also relates to new intermediate products and processes for their preparation.

It is already known that certain triazolopyrimidines possess fungicidal properties (cf. EP 0 550 113-A, WO 94-20 501, EP 0 613 900-A, U.S. Pat. No. 5,612,345-A, EP 0 834 513-A, WO 98-46 607 and WO 98-46 608). The efficiency of these substances is good but in some cases, leaves something to be desired when low quantities are used.

New triazolopyrimidines of the formula 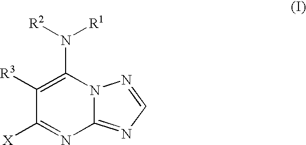
in which
R¹ represents optionally substituted alkyl, optionally substituted alkenyl, optionally substituted alkynyl, optionally substituted cyloalkyl or optionally substituted heterocyclyl,
R² represents hydrogen or alkyl or
R¹ and R² together with the nitrogen atom to which they are bound, represent an optionally substituted heterocyclic ring,
R³ represents optionally substituted pyridyl or optionally substituted pyrimidyl and
X represents halogen, have been found.

It has been further found that triazolopyrimidines of formula (I) can be prepared by reacting
(a) dihalogentriazolopyrimidines of the formula 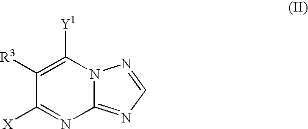
in which
R³ and X have the meanings specified above and
Y¹ represents halogen,
with amines of the formula 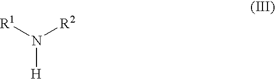
   in which
R¹ and R² have the meanings specified above,
optionally in the presence of a diluent, optionally in the presence of an acid acceptor and optionally in the presence of a catalyst.

Finally, it has been found that triazolopyrimidines of formula (I) are very well suited for combating undesirable micro-organisms. They primarily exhibit a strong fungicidal efficiency and can be used both for protecting plants and for protecting materials.

The triazolopyrimidines of formula (I) surprisingly have a substantially better microbicidal efficiency than the previously known substances having the same direction of action and the most similar constitution.

The compounds of formula (I) according to the invention can optionally be present as mixtures of various possible isomeric forms, especially of stereoisomers such as E, Z, threo and erythro isomers as well as optical isomers such as R and S isomers or atropisomers, but optionally also as tautomers.

Both the pure stereoisomers and also any mixtures of these isomers are the subject matter of this invention, even if compounds of formula (I) are only mentioned here in general.

According to the type of substituents defined above, the compounds of formula (I) have acid or basic properties and can form salts. If the compounds of formula (I) bear hydroxy, carboxy or other groups which induce acid properties, these compounds can be reacted with bases to form salts. Suitable bases, for example, are hydroxides, carbonates, hydrogen carbonates of alkali and alkaline-earth metals, especially those of sodium, potassium, magnesium and calcium, furthermore ammonia, primary, second and tertiary amines with (C₁-C₄) alkyl residues as well as mono-, di- and trialkanol amines of (C₁-C₄) alkanols. If the compounds of formula (I) bear amino, alkylamino or other groups which induce basic properties, these compounds can be reacts with acids to form salts. Suitable acids are, for example, mineral acids such as hydrochloric, sulphuric and phosphoric acid, organic acids such as acetic acid or oxalic acid and acid salts such as NaHSO₄ and KHSO₄. The salts thus obtained likewise show fungicidal and microbicidal properties.

The subject matter of the invention also comprises salt-like derivatives formed from compounds of formula (I) by reaction with basic or acid compounds as well as N-oxide which can be prepared by conventional oxygenation methods.

The triazolopyrimidines according to the invention are generally defined by formula (I). Preferred are those substances of formula (I) in which
R¹ represents alkyl with 1 to 6 carbon atoms which can be homogeneously or heterogeneously substituted between one and five times, by halogen, cyano, hydroxy, alkoxy with 1 to 4 carbon atoms and/or cycloalkyl with 3 to 6 carbon atoms, or
R¹ represents alkenyl with 2 to 6 carbon atoms which can be homogeneously or heterogeneously substituted between one and three times, by halogen, cyano, hydroxy, alkoxy with 1 to 4 carbon atoms and/or cycloalkyl with 3 to 6 carbon atoms, or
R¹ represents alkynyl with 3 to 6 carbon atoms which can be homogeneously or heterogeneously substituted between one and three times, by halogen, cyano, hydroxy, alkoxy with 1 to 4 carbon atoms and/or cycloalkyl with 3 to 6 carbon atoms, or
R¹ represents cycloalkyl with 1 to 6 carbon atoms which can be homogeneously or heterogeneously substituted between one and three times, by halogen and/or alkyl with 1 to 4 carbon atoms, or
R¹ represents saturated or unsaturated heterocyclyl with 5 or 6 ring members and 1 to 3 heteroatoms such as nitrogen, oxygen and/or sulphur, wherein the heterocyclyl can be substituted once or twice by halogen, alkyl with 1 to 4 carbon atoms, cyano and/or cycloalkyl with 3 to 6 carbon atoms,
R² represents hydrogen or alkyl with 1 to 4 carbon atoms or
R¹ and R² together with the nitrogen atoms to which they are bound, represent a saturated or unsaturated heterocyclic ring with 3 to 6 ring members, wherein the heterocyclic ring can contain a further nitrogen, oxygen or sulphur atom as ring member and wherein the heterocyclic ring can be optionally substituted up to three times by fluorine, chlorine, bromine, alkyl with 1 to 4 carbon atoms and/or haloalkyl with 1 to 4 carbon atoms and 1 to 9 fluorine and/or chlorine atoms,
R³ represents pyridyl which can be homogenously or heterogeneously substituted between one and four times by
   fluorine, chlorine, bromine, cyano,
   alkyl, alkoxy, hydroximinoalkyl or alkoximinoalkyl with respectively 1 to 3 carbon atoms,
   haloalkyl or haloalkoxy with respectively 1 to 3 carbon atoms
   or
R³ represents pyrimidyl which can be homogeneously or heterogeneously substituted between one and three times by
   fluorine, chlorine, bromine, cyano,
   alkyl, alkoxy, hydroximinoalkyl or alkoximinoalkyl with respectively 1 to 3 carbon atoms,
   haloalkyl or haloalkoxy with respectively 1 to 3 carbon atoms and 1 to 7 halogen atoms
   and
X represents fluorine, chlorine or bromine.

Especially preferred are those triazolopyrimidines of formula (I), in which
R¹ represents a residue of the formula 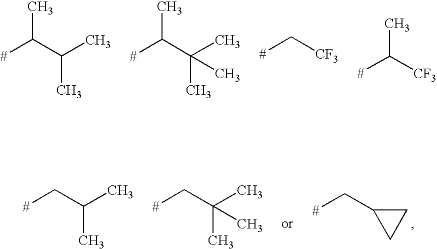
   wherein # marks the linking point, or
R¹ represents allyl, dichlorallyl, propargyl, cyclopropyl, cyclopentyl, cyclohexyl, piperidinyl or morpholinyl,
R² represents hydrogen, methyl or ethyl, or
R¹ and R² together with the nitrogen atom to which they are bound, represents pyrrolidinyl, piperidinyl, morpholinyl, thiomorpholinyl, piperazinyl, 3,6-dihydro-1(2H)-piperidinyl or tetrahydro-1(2H)-pyridazinyl, wherein these residues can be substituted by 1 to 3 fluorine atoms, 1 to 3 methyl groups and/or trifluoromethyl,
R³ represents pyridyl which is linked in the 2- or 4-position and can be homogeneously or heterogeneously substituted between one and four times by fluorine, chlorine, bromine, cyano, methyl, ethyl, methoxy, methylthio, hydroximinomethyl, hydroximinoethyl, methoximinomethyl, methoximinoethyl and/or trifluoromethyl, or
R³ represents pyrimidyl which is linked in the 4-position and can be homogeneously or heterogeneously substituted between one and three times by fluorine, chlorine, bromine, cyano, methyl, ethyl, methoxy, methylthio, hydroximinomethyl, hydroximinoethyl, methoximinomethyl, methoximinoethyl and/or trifluoromethyl,
   and
X represents fluorine or chlorine.

The aforementioned residue definitions can be arbitrarily combined amongst one another. In addition, individual definitions can be omitted.

If 5,7-dichloro-6-(5-chlorpyrimidin-4-yl)-[1,2,4]triazolo[1,5-a]pyrimidine and 2,2,2-trifluoro-isopropylamine are used as initial substances, the course of the method (a) according to the invention can be illustrated by the following formula scheme

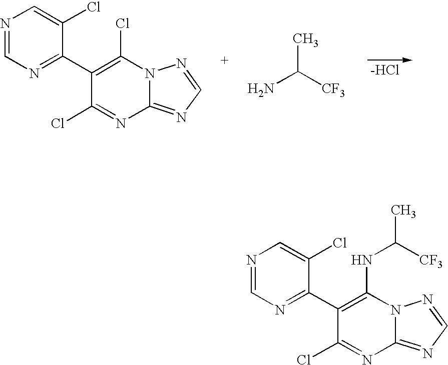

The dihalogen-triazolo-pyrimidines required as initial substances when implementing the method (a) according to the invention are generally defined by formula (II). In this formula (II) R³ and X preferably have the same meanings as were specified previously as preferred for these residues in connection with the description of the substances of formula (I) according to the invention. Y¹ preferably represents fluorine, chlorine or bromine, especially preferably fluorine or chlorine.

The dihalogen-triazolo-pyrimidines of formula (II) are new. These substances are also suitable for combating undesirable micro-organisms.

These dihalogen-triazolo-pyrimidines can be prepared by reacting
(b) dihydroxy-triazolo-pyrimidines of the formula 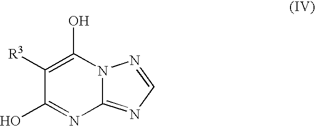
   in which
R³ has the meaning specified above
   with halogenating agents, optionally in the presence of a diluent.

If 6-(5-chloropyrimidin-4-yl)-[1,2,4]triazolo[1,5-a]-pyrimidin-5,7-diole is used as the initial substance and phosphorus oxychloride mixed with phosphorus pentachloride is used as the halogenating agent, the course of the method (b) according to the invention can be illustrated by the following formula scheme

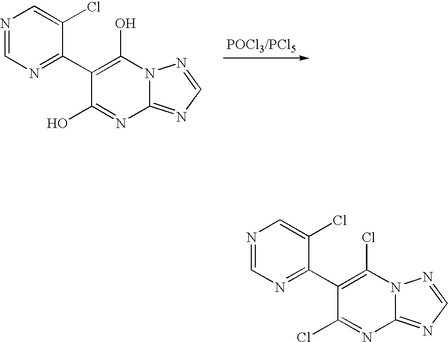

The dihydroxy-triazolopyrimidines required as initial substances when implementing the method (b) according to the invention, are generally defined by formula (IV). In this formula R³ preferably has the same meanings as were specified previously as preferred for this residue in connection with the description of the substances of formula (I) according to the invention

The dihydroxy-triazolo-pyrimidines of the formula (IV) were not known previously. They can be prepared by reacting
(c) heteroaryl malonic esters of the formula 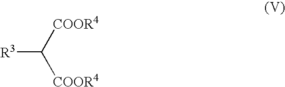
   in which
R³ has the meaning specified above and
R⁴ represents alkyl with 1 to 4 carbon atoms,
   with aminotriazole of the formula 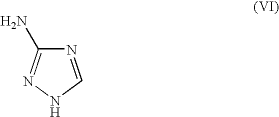
      optionally in the presence of a diluent and optionally in the presence of an acid binder.

If 2-(5-chloropyrimidin-4-yl)-malonic acid dimethylester and 3-aminotriazole are used as initial substances, the course of the method (c) according to the invention can be illustrated by the following formula scheme

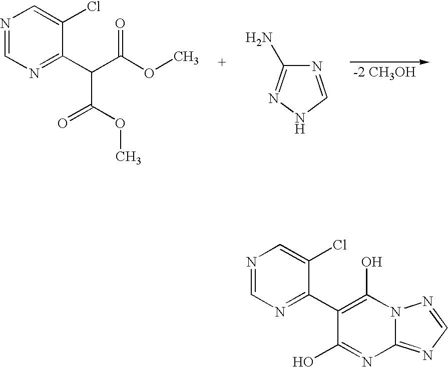

The heteroaryl malonic esters required as initial substances when implementing the method (c) according to the invention, are generally defmed by formula (V). In this formula R³ preferably has the same meanings as were specified previously as preferred for this residue in connection with the description of the substances of formula (I) according to the invention. R⁴ represents methyl or ethyl.

The heteroarylmalone esters of formula (V) are partly known (cf. DE 38 20 538-A and WO 01-11 965).

Pyridyl malonic esters of the formula 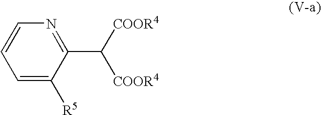
in which
R⁴ has the meaning specified above and
R⁵ represents halogen or haloalkyl, are new.

Also new are pyrimidyl malonic esters of the formula 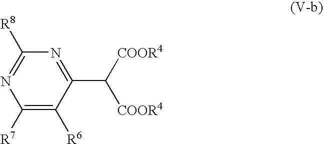
in which
R4 has the meaning specified above,
R⁶ represents halogen or haloalkyl and
R⁷ and R⁸ independently of one another represent hydrogen, fluorine, chlorine, bromine, methyl, ethyl or methoxy.

Pyridyl malonic esters of the formula (V-a) can be prepared by reacting
(d) pyridine halides of the formula 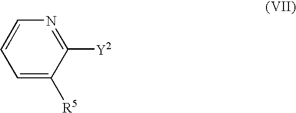
   in which
R⁵ has the meaning specified above and
Y² represents halogen,
   with malonic esters of the formula 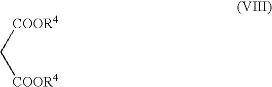
      in which
R⁴ has the meaning specified above,
   optionally in the presence of a diluent, optionally in the presence of a copper salt and optionally in the presence of an acid acceptor.

If 2-chloro-3-trifluormethylpyridine and malonic acid dimethylester are used as initial substances, the course of the method (d) according to the invention can be illustrated by the following formula scheme

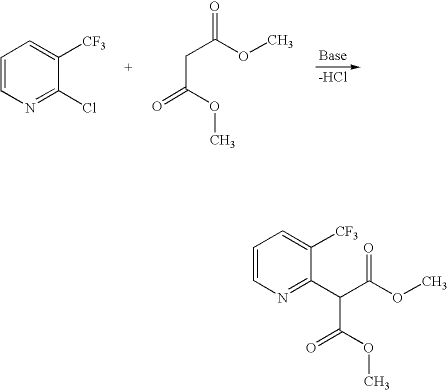

The pyridine halides required as initial substances for implementing the method (d) according to the invention, are generally defmed by formula (VII). In this formula R⁵ preferably represents fluorine, chlorine or trifluoromethyl. Y² preferably represents chlorine or bromine.

The pyridine halides of formula (VII) are known synthesis chemicals.

The malonic acid esters of formula (VIII) further required as initial substances for implementing the method (d) according to the invention are also known synthesis chemicals.

The pyrimidyl malonic esters of the formula (V-b) can be prepared by reacting
(e) pyrimidine halides of the formula 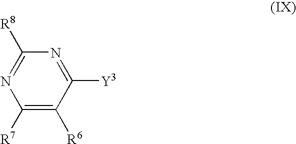
   in which
R⁶, R⁷ and R⁸ have the meanings specified above and
Y³ represents halogen,
   with malonic esters of the formula 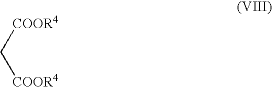
      in which
R⁴ has the meaning specified above,
   optionally in the presence of a diluent, optionally in the presence of a copper salt and optionally in the presence of an acid acceptor.

If 4,5-dichloropyrimidine and malonic acid dimethylester are used as initial substances, the course of the method (e) according to the invention can be illustrated by the following formula scheme.

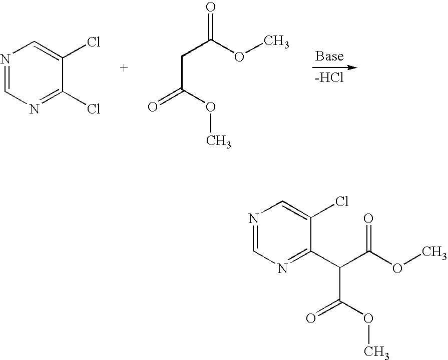

The pyrimidine halides required as initial substances for implementing the method (e) according to the invention, are generally defined by formula (IX). In this formula R⁶ preferably represents fluorine, chlorine or trifluoromethyl. R⁷ and R⁸ preferably represent, independently of one another, hydrogen, fluorine, chlorine, bromine, methyl, ethyl or methoxy. Y³ preferably represents chlorine or bromine.

The pyrimidine halides of formula (IX) are known can be prepared by known methods (cf. J. Chem. Soc. 1955, 3478, 3481).

The aminotriazole of formula (VI) further required as an initial substance for implementing the method (c) is a commercially available chemical.

All components usual for replacing hydroxy groups by halogen can be considered as halogenating agents for carrying out the method (b). Preferably used are phosphorus trichloride, phosphorus tribromide, phosphorus pentachloride, phosphorus oxychloride, thionyl chloride, thionyl bromide or mixtures thereof. The corresponding fluorine compounds of formula (II) can be prepared from the chlorine or bromine compounds by reacting with potassium fluoride.

Said halogenating agents are known.

The amines further required as initial substances for carrying out the method (a) according to the invention are generally defined by formula (III). In this formula R¹ and R² preferably have the same meanings which were given as preferred for R¹ and R² in connection with the description of the compounds of formula (I) according to the invention.

The amines of formula (III) are known or can be prepared using known methods.

All usual inert organic solvents can be considered as diluents for implementing the method (a) according to the invention. Preferably used are halogenated hydrocarbons such as, for example, chlorobenzene, dichlorobenzene, dichloromethane, chloroform, tetrachloromethane, dichloroethane or trichloroethane; ethers such as diethylether, diisopropylether, methyl-t-butylether, methyl-t-amylether, dioxane, tetrahydrofuran, 1,2-dimethoxyethane, 1,2-diethoxyethane or anisol; nitriles such as acetonitrile, propionitrile, n- or i-butyronitrile or benzonitrile; amides such as N,N-dimethylformamide, N,N-dimethylacetamide, N-methylformanilide, N-methylpyrrolidone or hexamethylphosphoric acid triamide; esters such as methyl acetate or ethyl acetate; sulphoxides such as dimethylsulphoxide; sulphones such as sulpholane.

All usual inorganic or organic bases for such reactions can be considered as acid acceptors for carrying out the method (a) according to the invention. Preferably used are alkaline-earth metal or alkali metal hydrides, hydroxides, amides, alcoholates, acetates, carbonates or hydrogen carbonates such as for example, sodium hydride, sodium amide, lithium diisopropylamide, sodium methylate, sodium ethylate, potassium tert-butylate, sodium hydroxide, potassium hydroxide, sodium acetate, potassium acetate, calcium acetate, sodium carbonate, potassium carbonate, potassium hydrogen carbonate and sodium hydrogen carbonate, and in addition ammonium compounds such as ammonium hydroxide, ammonium acetate and ammonium carbonate, as well as tertiary amines such as trimethylamine, triethylamine, tributylamine, N,N-dimethylaniline, N,N-dimethyl benzylamine, pyridine, N-methylpiperidine, N-methylmorpholine, N,N-dimethylaminopyridine, diazabicyclooctane (DABCO), diazabicyclononene (DBN) or diazabicycloundecene (DBU).

All reaction accelerators usual for such reactions can be considered as catalysts for carrying out the method (a) according to the invention. Preferably used are fluorides such as sodium fluoride, potassium fluoride or ammonium fluoride.

The reaction temperatures can be varied over a fairly large range for carrying out the method (a) according to the invention. Temperatures between 0° C. and 150° C., preferably temperatures between 0° C. and 80° C. are generally used.

For carrying out the method (a) according to the invention, generally 0.5 to 10 mol, preferably 0.8 to 2 mol of amine of formula (III) is used per 1 mol of dihalogen triazolo pyrimidine. Preparation is carried out by the usual methods.

All solvents usual for such halogenations can be considered as diluents for carrying out the method (a) according to the invention. Preferably used are halogenated aliphatic or aromatic hydrocarbons such as chlorobenzene. However, halogenating agents themselves, e.g. phosphorus oxychloride or a mixture of halogenating agents can also function as diluents.

The temperatures can also be varied over a fairly wide range when implementing method (b). In general, temperatures between 0° C. and 150° C., preferably between 10° C. and 120° C. are used.

When implementing method (b), dihydroxy-triazolpyrimidine of formula (IV) is generally reacted with an excess of halogenating agent. Preparation is carried out by the usual methods.

All inert organic solvents usual for these reactions can be considered as diluents for carrying out method (c). Preferably used are alcohols such as methanol, ethanol, n-propanol, i-propanol, n-butanol and tert-butanol.

All inorganic and organic bases usual for these reactions can be considered as acid binders for carrying out method (c). Preferably used are tertiary amines such as tributylamine or pyridine. Amine used in excess can also act as a diluent.

The temperatures can be varied over a fairly wide range when implementing method (c). In general, temperatures between 20° C. and 200° C., preferably between 50° C. and 180° C. are used.

Heteroarylmalonic esters of formula (V) and aminotriazoles of formula (VI) are generally reacted in equivalent quantities when carrying out method (c). However, it is also possible to use one or the other component in excess. Preparation is carried out by the usual methods.

All usual inert organic solvents can be considered as diluents when implementing methods (d) and (e) according to the invention. Preferably used are halogenated hydrocarbons such as, for example, chlorobenzene, dichlorobenzene, dichloromethane, chloroform, tetrachloromethane, dichloroethane or trichloroethane; ethers such as diethylether, diisopropylether, methyl-t-butylether, methyl-t-amylether, dioxan, tetrahydrofuran, 1,2 dimethoxyethane, 1,2-diethoxyethane or anisol; nitriles such as acetonitrile, propionitrile, n- or i-butyronitrile or benzonitrile; amides such as N,N-dimethylformamide, N,N-dimethylacetamide, N-methylformanilide, N-methylpyrrolidone or hexamethylphosphoric acid triamide; sulphoxides such as dimethylsulphoxide; sulphones such as sulpholane; alcohols such as methanol, ethanol, n- or i-propanol, n-, i-, sec- or tert-butanol, ethanediol, propane-1,2-diol, ethoxyethanol, methoxyethanol, diethyleneglycolmonomethylether, diethyleneglycolmonoethylether, mixtures thereof with water or also pure water.

The usual copper salts can be considered as copper salts in each case when implementing methods (d) and (e) according to the invention. Preferably used are copper(I) chloride or copper(I) bromide.

The usual inorganic or organic bases can be considered in each case as acid acceptors when implementing methods (d) and (e) according to the invention. Preferably used are alkaline-earth metal or alkali metal hydrides, hydroxides, amides, alcoholates, acetates, carbonates or hydrogen carbonates such as, for example, sodium hydride, sodium amide, lithium diisopropylamide, sodium methylate, sodium ethylate, potassium tert-butylate, sodium hydroxide, potassium hydroxide, sodium acetate, potassium acetate, calcium acetate, sodium carbonate, potassium carbonate, potassium hydrogen carbonate and sodium hydrogen carbonate and in addition ammonium compounds such as ammonium hydroxide, ammonium acetate and ammonium carbonate as well as tertiary amines such as trimethylamine, triethylamine, tributylamine, N,N-dimethylaniline, N,N-dimethylbenzylamine, pyridine, N-methylpiperidine, N-methylmorpholine, N,N-dimethylamino-pyridine, diazabicyclooctane (DABCO), diazabicyclononene (DBN) or diazabicycloundecene (DBU).

The reaction temperatures can be varied over a fairly wide range when implementing methods (d) and (e) according to the invention. In general, temperatures between 0° C. and 150° C., preferably temperatures between 0° C. and 80° C. are used.

When implementing method (d) according to the invention, 1 to 15 mol, preferably 1.3 to 8 mol of malonic ester of formula (VIII) is generally used per 1 mol of pyridine halide of formula (VII). Preparation is carried out by the usual methods.

When implementing method (e) according to the invention, 1 to 15 mol, preferably 1.3 to 8 mol of malonic ester of formula (VIII) is generally used per 1 mol of pyrimidine halide of formula (IX). Preparation is carried out by the usual methods.

The methods according to the invention are generally carried out at atmospheric pressure. However it is also possible to work at elevated pressure.

The substances according to the invention have a strong microbicidal effect and can be used to combat undesirable micro-organisms such as fungi and bacteria, to protect plants and to protect materials.

Fungicides can be used for plant protection to combat plasmodiophoromycetes, oomycetes, chytri-diomycetes, zygomycetes, ascomycetes, basidiomycetes and deuteromycetes.

Bactericides can be used in plant protection to combat Pseudomonadaceae, Rhizobiaceae, Enterobacteriaceae, Corynebacteriaceae and Streptomycetaceae.

Some pathogens of fungal and bacterial diseases which come within the above-mentioned headings may be mentioned as examples but are not restricted thereto:
Xanthomonas species such as, for example, Xanthomonas campestris pv. oryzae;
Pseudomonas species such as, for example, Pseudomonas syringae pv. lachrymans;
Erwinia species such as, for example, Erwinia amylovora;
Pythium species such as, for example, Pythium ultimum;
Phytophthora species such as, for example, Phytophthora infestans;
Pseudoperonospora species such as, for example, Pseudoperonospora humuli or
Pseudoperonospora cubensis;
Plasmopara species such as, for example, Plasmopara viticola;
Bremia species such as, for example, Bremia lactucae;
Peronospora species such as, for example, Peronospora pisi or P. brassicae;
Erysiphe species such as, for example, Erysiphe graminis;
Sphaerotheca species such as, for example, Sphaerotheca fuliginea;
Podosphaera species such as, for example, Podosphaera leucotricha;
Vent uria species such as, for example, Venturia inaequalis;
Pyrenophora species such as, for example, Pyrenophora teres or P. graminea (conodia form: Drechslera, Syn: Helminthosporium);
Cochliobolus species such as, for example, Cochliobolus sativus (conodia form: Drechslera, Syn: Helminthosporium);
Uromyces species such as, for example, Uromyces appendiculatus;
Puccinia species such as, for example, Puccinia recondita;
Scierotinia species such as, for example, Sclerotinia sclerotiorum;
Tilletia species such as, for example, Tilletia caries;
Ustilago species such as, for example, Ustilago nuda or Ustilago avenae;
Pellicularia species such as, for example, Pellicularia sasakii;
Pyricularia species such as, for example, Pyricularia oryzae;
Fusarium species such as, for example, Fusarium culmorum;
Botrytis species such as, for example, Botrytis cinerea;
Septoria species such as, for example, Septoria nodorum;
Leptosphaeria species such as, for example, Leptosphaeria nodorum;
Cercospora species such as, for example, Cercospora canescens;
Alternaria species such as, for example, Alternaria brassicae;
Pseudocercosporella species such as, for example, Pseudocercosporella herpotrichoides.

The active substances according to the invention have a very good strengthening effect in plants. They are thus suited for mobilising the plant's own defences against attack by undesirable micro-organisms.

Plant-strengthening (resistance-inducing) substances are to be understood in the present connection as those substances which are capable of stimulating the defence system of plants so that when inoculated subsequently with undesirable micro-organisms, the treated plants develop extensive resistance to these micro-organisms.

Undesirable micro-organisms are to be understood in the present case as phytopathogenic fungi, bacteria and viruses. The substances according to the invention can thus be used to protect plants from attack by said pathogens over a certain time following treatment. The period of time within which protection is afforded generally extends from 1 to 10 days, preferably 1 to 7 days after treatment of the plants with the active substances.

The good phytotolerance of the active substances in the concentrations necessary to combat plant diseases means that above-ground parts of plants, plants and seeds and the soil can be treated.

In this situation, the active substances according to the invention can be used particularly successfully to combat cereal diseases such as Erysiphe species, diseases in wine-growing, fruit and vegetable growing such as Botrytis, Venturia, Sphaerotheca and Podosphaera species.

The active substances according to the invention are suitable for increasing the crop yield. They are also less toxic and exhibit good phytotolerance.

The active substances according to the invention can optionally be used in certain concentrations and quantities as herbicides, to influence plant growth and also to combat animal pests. They can also optionally be used as intermediate and initial products for synthesising further active substances.

All plants and parts of plants can be treated according to the invention. In this context, plants are understood as all plants and plant populations such as desired and undesired wild plants and cultivated plants (including naturally occurring cultivated plants). Cultivated plants can be plants which can be obtained by conventional cultivation and optimisation methods or by biotechnology and gene technology methods or combinations of these methods, including transgenic plants and including plant varieties which can or cannot be protected by variety property rights. Parts of plants should be understood as above-ground and underground parts and organs of plants such as scion, leaf, flower and root, with leaves, needles, stakes, stems, flowers, fruiting bodies, fruits and seeds as well as roots, bulbs and rhizomes being cited as examples. Parts of plants also include harvested material as well as vegetative and generative replicative material, for example, cuttings, bulbs, rhizomes, scions and seeds.

The treatment according to the invention of plants and parts of plants with the active substances is carried out directly or by acting on their surroundings, habitat or storage area using the conventional treatment methods, e.g. by dipping, spraying, vaporising, nebulising, scattering, sprinkling and in the case of replicative material, especially in seeds, furthermore by single- or multilayer coating.

For the protection of materials, the substances according to the invention can be used to protect technical materials against attack and destruction by undesirable micro-organisms.

Technical materials are to be understood in the present connection as non-living materials which have been prepared for use in technology. For example, technical materials which are to be protected from microbial modification or destruction by active substances according to the invention are adhesives, glues, paper and cardboard, textiles, leather, wood, coating agents and plastic articles, cooling lubricants and other materials which can be attacked or destroyed by micro-organisms. Within the scope of materials to be protected, mention may also be made of parts of production installations, for example, cooling water circuits which can be adversely affected by multiplication of micro-organisms. Within the scope of the present invention, technical materials are preferably adhesives, glues, paper and cardboard, leather, wood, coating agents, cooling lubricants and heat-transfer liquids, especially preferably wood.

As micro-organisms which can bring about a degradation or modification of technical materials, mention may be made, for example, of bacteria, fungi, yeasts algae and slime organisms. The active substances according to the invention preferably act against fungi, especially moulds, wood-staining and wood-destroying fungi (basidiomycetes) and against slime organisms and algae.

Micro-organisms of the following genus may be mentioned as examples:
Alternaria such as Alternaria tenuis,
Aspergillus such as Aspergillus niger,
Chaetomium such as Chaetomium globosum,
Coniophora such as Coniophora puetana,
Lentinus such as Lentinus tigrinus,
Penicillium such as Penicillium glaucum,
Polyporus such as Polyporus versicolor,
Aureobasidium such as Aureobasidium pullulans,
Sclerophoma such as Sclerophoma pityophila,
Trichorma such as Trichorma viride,
Escherichia such as Escherichia coli,
Pseudomonas such as Pseudomonas aeruginosa,
Staphylococcus such as Staphylococcus aureus.

Depending on their respective physical and/or chemical properties, the active substances can be converted into the usual formulations such as solutions, emulsions, suspensions, powders, foams, pastes, granules, aerosols, ultrafine encapsulations in polymer materials, and in coating compounds for seeds, as well as ULV cold and warm mist formulations.

These formulations are produced in a known fashion, e.g. by mixing the active substances with extenders, i.e. liquid solvents, pressurised liquefied gases and/or solid carriers, optionally using surfactants, i.e. emulsifiers and/or dispersants and/or foaming agents. If water is used as an extending agent, for example, organic solvents can also be used as auxiliary solvents. Possible liquid solvents are substantially: aromatic compounds such as xylene, toluene or alkylnaphthalene, chlorinated aromatic compounds or chlorinated aliphatic hydrocarbons such as chlorobenzenes chloroethylenes or methylenechloride, aliphatic hydrocarbons such as cyclohexane or paraffins, e.g. petroleum fractions, alcohols such as butanol or glycol as well as their ethers and esters, ketones such as acetone, methylethylketone, methylisobutylketone or cyclohexanone, strongly polar solvents such as dimethylformamide and dimethylsulphoxide as well as water. Liquefied gaseous extenders or carriers means those liquids which are gaseous at normal temperature and at normal pressure e.g. aerosol propellant gases such as halogen hydrocarbons as well as butane, propane, nitrogen and carbon dioxide. Possible solid carriers are: e.g. natural ground stone such as kaolin, clay, talc, chalk, quartz, attapulgite, montmorillonite or diatomataceous earth and synthetic ground stone such as highly disperse silicic acid, aluminium oxide and silicates. Possible solid carriers for granules are: e.g. broken and fractionated natural stone such as calcite, pumice, sepiolith, dolomite as well as synthetic granules of inorganic or organic dust as well as granules of organic material such as sawdust, coconut shells, maize cobs and tobacco stalks. Possible emulsifiers and/or foaming agents are: e.g. non-ionogenic and anionic emulsifiers such as polyoxyethylene fatty acid esters, polyoxyethylene fatty alcohol ethers e.g. alkylarylpolyglycolether, alkylsulphonates, alkylsulphates, arylsulphonates and protein hydrolysates. Possible dispersants are: e.g: lignin sulphite waste and methylcellulose.

Adhesives such as carboxymethylcellulose, natural and synthetic powder, granular or latex-form polymers such as gum arabic, polyvinylalcohol, polyvinylacetate, as well as natural phospholipids such as cephaline and lecithin, and synthetic phospholipids can be used in the formulations. Further additives can be mineral and vegetable oils

Dyes such as inorganic pigments, e.g. iron oxide, titanium oxide, ferrocyan blue and organic dyes such as alizarine, azo and metal phthalocyanine dyes and trace nutrients such as salts of iron, manganese, boron, copper, cobalt, molybdenum and zinc can be used.

The formulations generally contain between 0.1 and 95 weight percent of active substance, preferably between 0.5 and 90%.

The active substances according to the invention can be used as such or in their formulations also mixed with known fungicides, bactericides, acaricides, nematicides or insecticides in order to thus broaden the spectrum of action, for example, or avoid the development of resistance. In many cases, synergistic effects are achieved in this situation, i.e., the efficiency of the mixture is higher than the efficiency of the individual components.

The following compounds can be considered as mixing partners, for example:

Fungicides:

2-phenylphenol; 8-hydroxyquinolinsulphate;
acibenzolar-S-methyl; aldimorph; amidoflumet; ampropylfos; ampropylfos potassium; andoprim; anilazine; azaconazole; azoxystrobin;
benalaxyl; benodanil; benomyl; benthiavalicarb-isopropyl; benzamacril; benzamacril-isobutyl; bilanafos; binapacryl; biphenyl; bitertanol; blasticidin-S; bromuconazole; bupirimate; buthiobate; butylamine;
calcium-polysulphide; capsimycin; captafol; captan; carbendazim; carboxin; carpropamid; carvone; quinomethionate; chlobenthiazone; chlorfenazole; chloroneb; chlorothalonil; chlozolinate; clozylacon; cyazofamid; cyflufenamid; cymoxanil; cyproconazole; cyprodinil; cyprofuram;
Dagger G; debacarb; dichlofluanid; dichione; dichlorophen; diclocymet; diclomezine; dicloran; diethofencarb; difenoconazole; diflumetorim; dimethirimol; dimethomorph; dimoxystrobin; diniconazole; diniconazole-M; dinocap; diphenylamine; dipyrithione; ditalimfos; dithianon; dodine; drazoxolon;
edifenphos; epoxiconazole; ethaboxam; ethirimol; etridiazole;
famoxadone; fenamidone; fenapanil; fenarimol; fenbuconazole; fenfuram; fenhexamid; fenitropan; fenoxanil; fenpiclonil; fenpropidin; fenpropimorph; ferbam; fluazinam; flubenzimine; fludioxonil; flumetover; flumorph; fluoromide; fluoxastrobin; fluquinconazole; flurprimidol; flusilazole; flusulphamide; flutolanil; flutriafol; folpet; fosetyl-Al; fosetyl-sodium; fuberidazole; furalaxyl; furametpyr; furcarbanil; furmecyclox;
guazatine;
hexachlorobenzene; hexaconazole; hymexazol;
imazalil; imibenconazole; iminoctadine triacetate; iminoctadine tris(albesil; iodocarb; ipconazole; iprobenfos; iprodione;iprovalicarb; irumamycin; isoprothiolane; isovaledione;
kasugamycin; kresoxim-methyl;
mancozeb; maneb; meferimzone; mepanipyrim; mepronil; metalaxyl; metalaxyl-M; metconazole; methasulphocarb; methfuroxam; metiram; metominostiobin; metsulphovax; mildiomycin; myclobutanil; myclozolin;
natamycin; nicobifen; nitrothal-isopropyl; noviflumuron; nuarimol;
ofurace; orysastrobin; oxadixyl; oxolinic acid; oxpoconazole; oycarboxin; oxyfenthiin;
paclobutrazol; pefurazoate; penconazole; pencycuron; phosdiphen; phthalide; picoxystrobin; piperalin; polyoxins; polyoxorim; probenazole; prochloraz; procymidone; propamocarb; propanosine-sodium; propiconazole; propineb; proquinazid; prothioconazole; pyraclostrobin; pyrazophos; pyrifenox; pyrimethanil; pyroquilon; pyroxyfur; pyrrolnitrine;
quinconazole; quinoxyfen; quintozene;
simeconazole; spiroxamine; sulphur;
tebuconazole; tecloftalam; tecnazene; tetcyclacis; tetraconazole;. thiabendazole; thicyofen; thifluzamide; thiophanate-methyl; thiram; tioxymid; tolclofos-methyl; tolylfluanid; triadimefon; triadimenol; triazbutil; triazoxide; tricyclamide; tricyclazole; tridemorph; trifloxystrobin; triflumizole; triforine; triticonazole;
uniconazole;
validamycin A; vinclozolin;
zineb; ziram; zoxamide;
(2S)-N-[2-[4-[[3-(4-chlorophenyl)-2-propinyl]oxy]-3-methoxyphenyl]ethyl]-3-methyl-2-[(methylsulphonyl)amino]-butanamide;
1-(1-naphthalenyl)-1H-pyrrol-2,5-dione;
2,3,5,6-tetrachloro-4-(methylsulphonyl)-pyridine;
2-amino-4-methyl-N-phenyl-5-thiazolcarboxamide;
2-chloro-N-(2,3-dihydro-1,1,3-trimethyl-1H-inden-4-yl)-3-pyridine carboxamide;
3,4,5-trichloro-2,6-pyridine dicarbonitrile;
actinovates;
cis-1-(4-chlorophenyl)-2-(1H-1,2,4-triazol-1-yl)-cycloheptanol;
methyl 1-(2,3-dihydro-2,2-dimethyl-1H-inden-1-yl)-1H-imidazol-5-carboxylate;
monopotassium carbonate;
N-(6-methoxy-3-pyridinyl)-cyclopropane carboxamide;
sodium tetrathiocarbonate;
   as well as copper salts and preparations such as Bordeaux mixture; copper hydroxide; copper naphthenate; copper oxychloride; copper sulphate; cufraneb; copper oxide; mancopper; oxine-copper.
   Bactericides:

Bronopol, dichlorophen, nitrapyrin, nickel-dimethyldithiocarbamate, kasugamycin, octhilinon, furancarbonic acid, oxytetracyclin, probenazol, streptomycin, tecloftalam, copper sulphate and other copper preparations.

Insecticides/Acaricides/Nematicides:

Abamectin, ABG-9008, acephate, acequinocyl, acetamiprid, acetoprole, acrinathrin, AKD-1022, AKD-3059, AKD-3088, alanycarb, aldicarb, aldoxycarb, allethrin, allethrin 1R-isomers, alphacypermethrin (alphamethrin), amidoflumet, aminocarb, amitraz, avermectin, AZ-60541, azadirachtin, azamethiphos, azinphos-methyl, azinphos-ethyl, azocyclotin,
Bacillus popilliae, Bacillus sphaericus, Bacillus subtilis, Bacillus thuringiensis, Bacillus thuringiensis strain EG-2348, Bacillus thuringiensis strain GC-91, Bacillus thuringiensis strain NCTC-11821, Baculoviruses, Beauveria bassiana, Beauveria tenella, bendiocarb, benfuracarb, bensultap, benzoximate, beta-cyfluthrin, beta-cypermethrin, bifenazate, bifenthrin, binapacryl, bioallethrin, bioallethrin-S-cyclopentyl-isomer, bioethanomethrin, biopermethrin,. bioresmethrin, bistrifluron, bpmc, brofenprox, bromophos-ethyl, bromopropylate, bromfenvinfos (-methyl), BTG-504, BTG-505, bufencarb, buprofezin, butathiofos, butocarboxim, butoxycarboxim, butylpyridaben,
cadusafos, camphechlor, carbaryl, carbofuran, carbophenothion, carbosulphan, cartap, CGA-50439, quinomethionate, chlordane, chlordimeform, chloethocarb, chlorethoxyfos, chlorfenapyr, chlorfenvinphos, chlorfluazuron, chlormephos, chlorobenzilate, chloropicrin, chlorproxyfen, chlorpyrifos-methyl, chlorpyrifos (-ethyl), chlovaporthrin, chromafenozide, cis-cypermethrin, cis-resmethrin, cis-permethrin, clocythrin, cloethocarb, clofentezine, clothianidin, clothiazoben, codlemone, coumaphos, cyanofenphos, cyanophos, cycloprene, cycloprothrin, cydia pomonella, cyfluthrin, cyhalothrin, cyhexatin, cypermethrin, cyphenothrin (1R-trans-isomer), cyromazine,
DDT, deltamethrin, demeton-s-methyl, demeton-s-methylsulphon, diafenthiuron, dialifos, diazinon, dichlofenthion, dichlorvos, dicofol, dicrotophos, dicyclanil, diflubenzuron,. dimethoate, dimethylvinphos, dinobuton, dinocap, dinotefuran, diofenolan, disulphoton, docusat-sodium, dofenapyn, DOWCO-439,
eflusilanate, emamectin, emamectin-benzoate, empenthrin (1R-isomer), endosulphan, entomopthora spp., EPN, esfenvalerate, ethiofencarb, ethiprole, ethion, ethoprophos, etofenprox, etoxazole, etrimfos,
famphur, fenamiphos, fenazaquin, fenbutatin oxide, fenfluthrin, fenitrothion, fenobucarb, fenothiocarb, fenoxacrim, fenoxycarb, fenpropathrin, fenpyrad, fenpyrithrin, fenpyroximate, fensulphothion, fenthion, fentrifanil, fenvalerate, fipronil, flonicamid, fluacrypyrim, fluazuron, flubenzimine flubrocythrinate, flucycloxuron, flucythrinate, flufenerim, flufenoxuron, flufenprox, flumethrin, flupyrazofos, flutenzin (flufenzine), fluvalinate, fonofos, formetanate, formothion, fosmethilan, fosthiazate, fubfenprox (fluproxyfen), furathiocarb,
gamma-HCH, gossyplure, grandlure, granulose viruses,
halfenprox, halofenozide, HCH, HCN-801, heptenophos, hexaflumuron, hexythiazox, hydramethylnone, hydroprene,
IKA-2002, imidacloprid, imiprothrin, indoxacarb, iodofenphos, iprobenfos, isazofos, isofenphos, isoprocarb, isoxathion, ivermectin,
japonilure,
kadethrin, nuclear polyhedrosis viruses, kinoprene,
lambda-cyhalothrin, lindane, lufenuron,
malathion, mecarbam, mesulphenfos, metaldehyd, metam-sodium, methacrifos, methamidophos, metharhizium anisopliae, metharhizium flavoviride, methidathion, methiocarb, methomyl, methoprene, methoxychlor, methoxyfenozide, metolcarb, metoxadiazone, mevinphos, milbemectin, milbemycin, MKI-245, MON-45700, monocrotophos, moxidectin, MTI-800,
naled, NC-104, NC-170, NC-184, NC-194, NC-196, niclosamide, nicotine, nitenpyram, nithiazine, NNI-0001, NNI-0101, NNI-0250, NNI-9768, novaluron, noviflumuron,
OK-5101, OK-5201, OK-9601, OK-9602, OK-9701, OK-9802, omethoate, oxamyl, oxydemeton-methyl,
Paecilomyces fumosoroseus, parathion-methyl, parathion (-ethyl), permethrin (cis-, trans-), petroleum, PH-6045, phenothrin (1R-trans isomer), phenthoate, phorate, phosalone, phosmet, phosphamidon, phosphocarb, phoxim, piperonyl butoxide, pirimicarb, pirimiphos-methyl, pirimiphos-ethyl, prallethrin, profenofos, promecarb, propaphos, propargite, propetamphos, propoxur, prothiofos, prothoate, protrifenbute, pymetrozine, pyraclofos, pyresmethrin, pyrethrum, pyridaben, pyridalyl, pyridaphenthion, pyridathion, pyrimidifen, pyriproxyfen,
quinalphos,
resmethrin, RH-5849, ribavirin, RU-12457, RU-15525,
S-421, S-1833, salithion, sebufos, SI-0009, silafluofen, spinosad, spirodiclofen, spiromesifen, sulphluramid, sulphotep, sulprofos, SZI-121,
tau-fluvalinate, tebufenozide, tebufenpyrad, tebupirimfos, teflubenzuron, tefluthrin, temephos, temivinphos, terbam, terbufos, tetrachlorvinphos, tetradifon, tetramethrin, tetramethrin (1R-isomer), tetrasul, theta-cypermethrin, thiacloprid, thiamethoxam, thiapronil, thiatriphos, thiocyclam hydrogen oxalate, thiodicarb, thiofanox, thiometon, thiosultap-sodium, thuringiensin, tolfenpyrad, tralocythrin, tralomethrin, transfluthrin, triarathene, triazamate, triazophos, triazuron, trichlophenidine, trichlorfon, triflumuron, trimethacarb,
vamidothion, vaniliprole, verbutin, Verticillium lecanii,
WL-108477, WL-40027,
YI-5201, YI-5301, YI-5302,
XMC, xylylcarb,
ZA-3274, Zeta-Cypermethrin, Zolaprofos, ZXI-8901,
the compound 3-methyl-phenyl-propylcarbamate (Tsumacide Z),
the compound 3-(5-chloro-3-pyridinyl)-8-(2,2,2-trifluorethyl)-8-azabicyclo[3.2.1]octan-3-carbonitrile (CAS Reg. No. 185982-80-3) and the corresponding 3-endo-isomers (CAS Reg. No. 185984-60-5) (cf. WO-96/37494, WO-98/25923),
   as well as preparations containing plant extracts having an insecticide effect, nematodes, fungi or viruses.

A mixture with other known active substances such as herbicides or with feitilisers and growth regulators, safeners or semiochemicals is also possible.

The compounds of formula (I) according to the invention also exhibit very good antimycotic effects. They have a very broad antimycotic spectrum of action, especially against dermatophytes and yeast fungi, mould and diphasic fungi (e.g. towards Candida species such as Candida albicans, Candida glabrata) as well as Epidermophyton floccosum, Aspergillus species such as Aspergillus niger and Aspergillus fumigatus, Trichophyton species such as Trichophyton mentagrophytes, Microsporon species such as Microsporon canis and audouinii. The listing of these fungi in no way represents any restriction of the mycotic spectrum which can be covered but is merely of an explanatory nature.

In addition, the compounds of formula (I) according to the invention are also suitable for suppressing the growth of tumour cells in humans and mammals. This is based on an interaction of the compounds according to the invention with tubulin and microtubuli and by promoting microtubuli polymerisation.

An effective quantity of one or more compounds of formula (I) or pharmaceutically tolerable salts thereof can be administered for this purpose.

The active substances can be used as such, in the form of their formulations or forms of application derived therefrom such as ready-to-use solutions, suspensions, spray powder, pastes, soluble powders, dusting agents and granules. Application takes place in the usual manner e.g. by pouring, splashing, spraying, scattering, dusting, foaming, brushing etc. It is further possible to apply the active substances by the ultra-low volume method or to inject the active substance preparation or the active substance itself into the soil. The seeds of plants can also be treated.

When the active substances according to the invention are used as fungicides, the quantities used can be varied over a fairly large range according to the type of application. When parts of plants are treated, the quantities of active substance used are generally between 0.1 and 10,000 g/ha, preferably between 10 and 1000 g/ha. When seed is treated, the quantities of active substance used are generally between 0.001 and 50 g per kilogram of seed, preferably between 0.01 and 10 g per kilogram of seed. When the soil is treated, the quantities of active substance used are generally between 0.1 and 10,000 g/ha, preferably between 10 and 5000 g/ha.

As has already been mentioned above, all plants and parts thereof can be treated according to the invention. In a preferred embodiment plant species and plant varieties and parts thereof which occur in the wild or are obtained by conventional biological cultivation methods such as crossing or protoplast fusion are treated. In a further preferred embodiment transgenic plants and plant varieties and parts thereof obtained by gene technology methods (genetically modified organisms) optionally in conjunction with conventional methods are treated. The term “parts” or “parts of plants” or “plant parts” has been explained above.

It is especially preferable if plants of the respectively commercially available plant varieties or those in common use are treated according to the invention. Plant varieties is understood as plants with new properties (“traits”) which have been cultivated by conventional cultivation, by mutagenesis or by recombinant DNA techniques. These can be varieties, strains, biotypes or genotypes.

According to the plant species or plant varieties, their location and growth conditions (soils, climate, vegetation period, nourishment), superadditive (“synergistic”) effects can occur as a result of the treatment according to the invention. Thus, for example, it is possible to achieve reduced quantities used and/or extensions of the spectrum of action and/or an intensification of the action of the substances and means used according to the invention, better. plant growth, increased tolerance to high or low temperatures, increased tolerance to drought or towards water and soil salt content, increased flowering performance, easier harvesting, faster ripening, higher crop yields, higher quality and/or higher nutritional value of the harvest products, higher storage capacity and/or processability of the harvest products which go beyond the effects which are actually expected.

Preferred transgenic (obtained by gene technology) plants or plant varieties to be treated according to the invention include all plants obtained by gene technology modification of genetic material which imparts to these plants particularly advantageous valuable properties (“traits”). Examples of such properties are better plant growth, increased tolerance to high or low temperatures, increased tolerance to drought or towards water and soil salt content, increased flowering performance, easier harvesting, faster ripening, higher crop yields, higher quality and/or higher nutritional value of the harvest products, higher storage capacity and/or processability of the harvest products. Further and especially emphasised examples of such properties are an increased defence of the plants towards animal and microbial pests, and towards insects, mites, phytopathogenic fungi, bacteria and/or viruses and an increased tolerance of the plants towards specific herbicidal active substances. As examples of transgenic plants, mention is made of important cultivated plants such as cereals (wheat, rice), maize, soya, potato, cotton, tobacco, rape and fruit plants (with the fruits apples, pears, citrus fruits and grapes), with maize, soya, potato, cotton, tobacco and rape being particularly emphasised. Properties (“traits”) particularly emphasised include the increased defence of the plants toward insects, arachnids, nematodes and snails as a result of toxins produced in the plants, especially those produced in the plants (hereinafter called “Bt plants”) by the genetic material from Bacillus Thuringiensis (e.g. by the genes CryIA(a), CryIA(b), CryIA(c), CryIIA, CryIIIA, CryIIIB2, Cry9c Cry2Ab, Cry3Bb and CryIF and combinations thereof). Properties (“traits”) especially emphasised include the increased defence of plants towards fungi, bacteria, viruses through systemically acquired resistance (SAR), systemin, phytoalexins, elicitors as well as resistance genes and suitably expressed proteins and toxins. Properties (“traits”) especially emphasised include the increased tolerance of the plants toward certain herbicidal active substances, for example, imidazolinones, sulphonyl ureas, glyphosate or phosphinotricin (e.g. “PAT”-Gene). The genes imparting the desired properties (“traits”) in each case can also occur in combination with one another in transgenic plants. As examples of “Bt plants” mention may be made of maize varieties, cotton varieties, soya varieties and potato varieties which are sold under the tradenames YIELD GARD® (e.g. maize, cotton, soya), KnockOut® e.g. maize), StarLink® (e.g. maize), Bollgard® (cotton), Nucoton® (cotton) and NewLeaf® (potato). As examples of herbicide-tolerant plants mention may be made of maize varieties, cotton varieties and soya varieties which are sold under the tradenames Roundup Ready® (tolerance towards glyphosates e.g. maize, cotton, soya), Liberty Link® (tolerance towards phosphinotricin, e.g. rape), IMI® (tolerance towards imidazolinones) and STS® (tolerance towards suiphonyl ureas, e.g. maize). As herbicide-resistant (cultivated conventionally for herbicide tolerance) plants, mention may also be made of varieties (e.g. maize) sold under the name Clearfield®. Naturally these statements also apply to varieties of plants developed in the future or coming onto the market in the future having these genetic properties (“traits”) or those developed in the future.

The plants listed can especially advantageously be treated with compounds of the general formula (I) or active-substance mixtures according to the invention. The preferred ranges given above for the active substances or mixture also apply to the treatment of these plants. The treatment of plants with the compounds or mixtures specially mentioned in the present text is particularly emphasised.

The preparation and use of the active substances according to the invention is deduced from the following examples.

### PREPARATION EXAMPLES

### Example 1

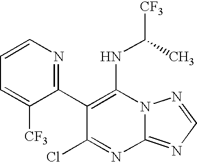 Method (a)

0.1 g of potassium fluoride is added to a solution of 0.3 g (0.9 mmol) of 5,7-dichloro-6-(3-trifluormethyl-pyridin-2 -yl)-[1,2,4]triazolo[1,5-a]pyrimidine in 10 ml acetonitrile, stirred for 2 hours at 80° C. and then cooled to 0° C. 0.21 g (1.9 mmol) of (S)-2,2,2-trifluoroisopropylamine is added to the solution and stirred for 18 hours at 80° C. The reaction mixture is added to 30 ml of 1N hydrochloric acid, stirred and extracted with dichloromethane. The organic phases is washed twice with water, dried over sodium sulphate and concentrated at reduced pressure. 0.23 g (60.4% of theoretical yield) of N-{5-chloro-6-[3-(trifluoromethyl)-2-pyridinyl][1,2,4]triazolo[1,5-a]pyrimidin-7-yl}-N-[(1 S)-2,2,2-trifluor-1-methylethyl]amine is obtained.
HPLC: log P=2.22

### Example 2

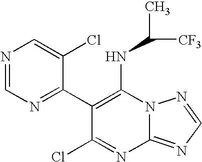 Method (a)

0.2 g of potassium fluoride is added to a solution of 0.5 g (1.66 mmol) of 5,7-dichloro-6-(5-chloro-4-pyrimidinyl)[1,2,4]triazolo[1,5-a]pyrimidine in 10 ml of acetonitrile, stirred for 2 hours at 80° C. and then cooled to room temperature. 0.375 g (3.32 mmol) of (S)-2,2,2-trifluorisopropylamine is added to the solution and stirred for 18 hours at 80° C. The reaction mixture is added to 30 ml of 1N hydrochloric acid, stirred and extracted with ethyl acetate. The organic phase is washed twice with water, dried over sodium sulphate and concentrated at reduced pressure. The residue is filtered with cyclohexane/ethyl acetate (3:1) over a short column of silica gel. 0.28 g (44.5% of theoretical yield) of N-[5-chloro-6-(5-chloro-4-pyrimidinyl)[1,2,4]triazolo[1,5-a]pyrimidin-7-yl]-N-[(1S)-2,2,2-trifluoro-1-methylethyl]amine is obtained.
HPLC: log P=1.98

The compounds of formula (I) given in the following Table 1 are also obtained using the methods specified above.

TABLE 1

| | |
|---|---|
| | (I) |
| 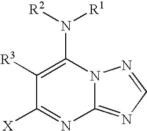 | |

| Ex. | | | | | | |
|---|---|---|---|---|---|---|
| No. | R¹ | R² | R³ | X | logP | Fp. (° C.): |
| 3 | i-Butyl | —H | 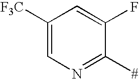 | —Cl | 2.85 | 133-4 |
| 4 | —CH²—C(CH₃)═CH₂ | —C₂H₅ | 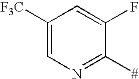 | —Cl | 3.51 | Paste |
| 5 | —NH—CH₂—CH₂—CH₂—CH₂— | | 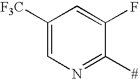 | —Cl | 2.52 | 168-9 |
| 6 | 2,2,2-trifluoro-1-methylethyl | —H | 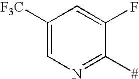 | —Cl | 2.91 | 175-6 |
| 7 | 1,2-dimethylpropyl | —H | 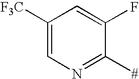 | —Cl | 3.13 | Paste |
| 8 | R-2,2,2-trifluoro-1-methylethyl | —H | 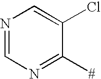 | —Cl | 1.98 | |
| 9 | —CH(CH₃)—C(CH₃)₃ | —H | 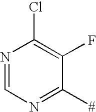 | —Cl | 3.14 | Paste |
| 10 | 1,2,2-trimethylpropyl | —H | 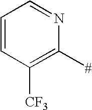 | —Cl | 2.8 | 195-7 |
| 11 | 1,2-dimethylpropyl | —H | 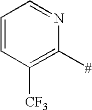 | —Cl | 2.53 | 153-5 |
| 12 | —CH(CH₃)—C(CH₃)₃ | —H | 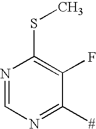 | —Cl | 3.28 | Paste |
| 13 | 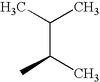 | —H | 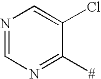 | —Cl | 2.16 | |
| 14 | 1,2,2-trimethylpropyl | —H | 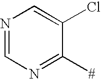 | —Cl | 2.51 | |
| 15 | 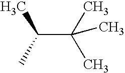 | —H | 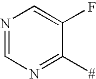 | —Cl | 2.37 | |
| 16 | 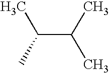 | —H | 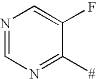 | —Cl | 2.04 | |
| 17 | 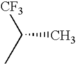 | —H | 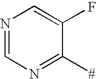 | —Cl | 1.87 | |
| 18 | 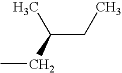 | —H | 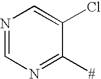 | —Cl | 2.22 | |
| 19 | —CH₂—CH₂—CH(CH₃)—CH₂—CH₂— | —H | 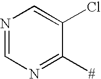 | —Cl | 2.39 | |

#indicates the linking point
*) The logP values were determined in accordance with EEC-Directive 79/831 Annex V. A8 by HPLC (Gradient method, acetonitrile/0.1% aqueous phosphoric acid)

Preparation of Initial Products of Formula (II)

### Example 20

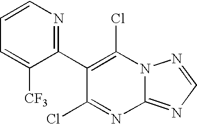 Method (b)

8 g (16 mmol) of 6-(3-trifluoromethyl-pyridin-2-yl)-[1,2,4]triazolo[1,5-a]pyrimidin-5,7-diole is mixed with 12 ml of phosphorus oxychloride. To this is added 2.7 g of phosphorus pentachloride in portions. The mixture is heated for 2 hours under reflux. After cooling, the reaction mixture is concentrated at reduced pressure, mixed with 100 ml of water and extracted three times with 100 ml of dichloromethane in each case. The combined organic phases are washed twice with 50 ml of water, dried over sodium sulphate and concentrated at reduced pressure. The residue is chromatographed with dichloromethane/methyl-t-butyl ether (95:5) on silica gel. 1.4 g (25.7% of theoretical yield) of 5,7-dichloro-6-(3-trifluoromethyl-pyridin-2-yl)-[1,2,4]triazolo[1,5-a]pyrimidine is obtained.
HPLC: log P=1.97

### Example 21

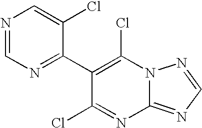 Method (b)

8 g (16 mmol) of 6-(5-chloro-4-pyrimidinyl)[1,2,4]triazolo[1,5-a]pyrimidin-5,7-diole is mixed with 25 ml of phosphorus oxychloride. 3.1 g of phosphorus pentachloride is added in portions. The mixture is stirred for 3 hours at 110° C. After cooling to room temperature, the reaction mixture is mixed with 300 ml of water and extracted three times with 100 ml of dichloromethane in each case. The combined organic phases are dried over sodium sulphate and concentrated at reduced pressure. The residue is chromatographed with hexane/ethyl acetate (9:1-5:1) on silica gel. 1.4 g (25.7% of theoretical yield) of 5,7-dichloro-6-(5-chloro-4-pyrimidinyl)[1,2,4]triazolo[1,5-a]pyrimidine is obtained.
HPLC: log P=1.43
   Preparation of Initial Products of Formula (IV)

### Example 22

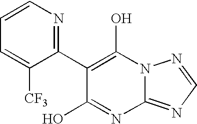 Method (c)

5.5 g (19.84 mmol) of 2-(3-trifluoromethyl-pyridin-2-yl)-malonic acid dimethylester and 1.67 g (19.84 mmol) of 3-amino-1,2,4-triazole are stirred in 5.2 ml of tributylamine for 2 hours at 180° C. The methanol produced during the reaction is continuously distilled off. After cooling, the desired product is separated from the tributylamine. The tributylamine is decanted off and the product 6-(3-trifluoromethyl-pyridin-2-yl)-[1,2,4]triazolo[1,5-a]pyrimidin-5,7-diole (yield: about 8 g, 60% purity) is used without further purification in the next reaction step.
HPLC: log P=−0.23

### Example 23

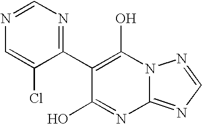 Method (c)

10 g (40.9 mmol) of 2-(5-chloro-4-pyrimidin-2-yl)-malonic acid dimethylester and 3.44 g (40.9 mmol) of 3-amino-1,2,4-triazole are stirred in 10.7 ml of tributylamine for 2 hours at 185° C. The methanol produced during the reaction is continuously distilled off. After cooling, the desired product is separated from the tributylamine. The tributylamine is decanted off and the product 6-(5-chloro-4-pyrimidinyl)[1,2,4]triazolo[1,5-a]pyrimidin-5,7-diole (yield: about 15 g, 11% purity, about 15% of theoretical yield) is used without further purification in the next reaction step.
HPLC: log P=−0.23
   Preparation of Initial Products of Formula (V-a)

### Example 24

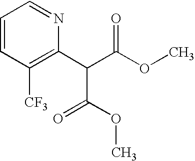 Method (d)

9 g (207 mmol) of 60% sodium hydride suspension is suspended in 300 ml of dioxane. To this 27.29 g (206.6 mmol) of malonic acid dimethylester is added dropwise at 55-60° C. and stirred for a further 30 minutes at the same temperature. After adding 8.18 g (82.63 mmol) of copper(I) chloride, the mixture is heated to 80° C. and then 15 g (82.63 mmol) of 2-chloro-3-trifluormethylpyridine is added dropwise. The reaction mixture is now stirred for another 14 hours at 100° C. After subsequent cooling to 15-20° C., concentrated hydrochloric acid is slowly added dropwise until the mixture shows an acidic reaction. Now 600 ml of water and 300 ml of dichloromethane are added and insoluble components are filtered off. The organic phase is separated from the filtrate, dried over sodium sulphate and concentrated at reduced pressure. The residue is chromatographed with hexane/ethyl acetate (4:1) on silica gel. 10.1 g (40% of theoretical yield) of 2-[3-trifluoromethyl]-pyrimidin-2-yl)-malonic acid dimethylester is obtained.
HPLC: log P=2.05
   Preparation of Initial Products of Formula (V-b)

### Example 25

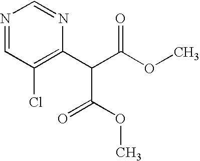 Method (e)

2.6 g (65.4 mmol) of 60% sodium hydride suspension is suspended in 100 ml of tetrahydrofuran. To this is added 6.9 g (52.4 mmol) of malonic acid dimethylester at 0° C. and stirred for 0.5 hours at the same temperature. A solution of 6.5 g (43.63 mMol) of 4,5-dichloropyrimidine is then added dropwise into 50 ml of tetrahydrofuran and stirred for a further 3 hours at room temperature. 150 ml of 1N hydrochloric acid is then added dropwise and then extracted with 100 ml of dichloromethane. The organic phase is separated, dried over sodium sulphate and concentrated at reduced pressure. The residue is chromatographed with methyl-t-butyl ether/petroleum ether (1:9) on silica gel. 7 g (65.6% of theoretical yield) of 2-(5-chloro-4-pyrimidin-2-yl)-malonic acid dimethylester is obtained.
HPLC: log P=1.33
   Preparation of 4,5-dichloroynrimidine

### Example 26

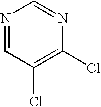

1.6 ml of dimethylamine is added to a solution of 112.5 g (673.7 mmol) of 5-chloro-6-oxo-1,6-dihydropyrimidin-1-ium chloride in 630 ml phosphorus oxychloride and heated for 3 hours under reflux. The excess phosphorus oxychloride is then distilled off at reduced pressure. After cooling, the residue is poured onto 1.5 l of iced water and extracted with 500 ml of dichloromethane, the organic phase is dried over sodium sulphate and concentrated at reduced pressure. 72.3 g (66.3% of theoretical yield) of 4,5-dichloropyrimidine is obtained.
HPLC: logP=1.35
   Preparation of 5-chloro-6-oxo-1,6-dihydropyrimidin-1-ium Chloride

### Example 27

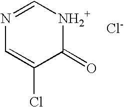

6.5 g (40 mmol) of iron-III-chloride is added to a solution of 77 g (0.8 mol) of 4(3H)-pyrimidinone in 770 ml of glacial acetic acid and 113.6 g (1.6 Mol) of chlorine is introduced at 40-45° C. The reaction mixture is cooled to 15° C., the solid product obtained is extracted and washed with ether. 112.5 g (84% of theoretical yield) of 5-chloro-6-oxo-1,6-dihydropyrimidin-1-ium chloride is obtained.

Preparation of 4(3H)-pyrimidinone

### Example 28

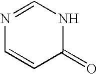

A mixture of 103 g (0,804 mol) 6-mercapto-4(1H)-pyrimidinone (JP 50053381, Chein. Abstr. CAN 84:17404) and 141.5 g (1.2 mol) of Raney nickel in 1.2 l ethanol is heated for 8 hours under reflux. The solution is hot-filtered, the residue washed with ethanol and the filtrate concentrated at reduced pressure. 67.2 g (87% of theoretical yield) of 4(3H)-pyrimidinone is obtained.

### Examples of Usage

### Example A

Podosphaera test (apple)/protective

| | |
|---|---|
| Solvent: | 24.5 parts by weight of acetone |
| | 24.5 parts by weight of dimethylacetamide |
| Emulsifier: | 1 part by weight of alkyl-aryl-polyglycolether |

One part by weight of active substance is mixed with the specified quantities of solvent and emulsifier and the concentrate diluted with water to the desired concentration to prepare a suitable active substance preparation.

To test the protective efficiency, young plants are sprayed with the active substance preparation in the specified amount. After drying the spray coating, the plants are inoculated with an aqueous spore suspension of the apple powdery mildew pathogen Podosphaera leucotricha. The inoculated plants are then placed in a glasshouse at about 23° C. and about 70% relative humidity.

The assessment is made 10 days after the inoculation. In this situation 0% means an efficiency corresponding to that of the control whereas an efficiency of 100% means that no attack is observed.

In this test the substances according to the invention given in Examples 1, 2, 8, 10, 11, 13, 14, 15, 16 and 17 show an efficiency higher than 90% when used in a quantity of 100 g/ha.

### Example B

Venturia test (apple)/protective

| | |
|---|---|
| Solvent: | 24.5 parts by weight of acetone |
| | 24.5 parts by weight of dimethylacetamide |
| Emulsifier: | 1 part by weight of alkyl-aryl-polyglycolether |

One part by weight of active substance is mixed with the specified quantities of solvent and emulsifier and the concentrate diluted with water to the desired concentration to prepare a suitable active substance preparation.

To test the protective efficiency, young plants are sprayed with the active substance preparation in the specified amount. After drying the spray coating, the plants are inoculated with an aqueous conidia suspension of the apple scab pathogen Venturia inaequalis and then left for one day at about 20° C. and 100% relative humidity in an incubation cabinet.

The plants are then placed in a greenhouse at about 21° C. and about 90% relative humidity.

The assessment is made 10 days after the inoculation. In this situation 0% means an efficiency corresponding to that of the control whereas an efficiency of 100% means that no attack is observed.

In this test the substances according to the invention given in Examples 2, 13, 14, 15, 16 and 17 show an efficiency higher than 90% when used in a quantity of 100 g/ha.

### Example C

Botrytis test (bean)/protective

| | |
|---|---|
| Solvent: | 24.5 parts by weight of acetone |
| | 24.5 parts by weight of dimethylacetamide |
| Emulsifier: | 1 part by weight of alkyl-aryl-polyglycolether |

One part by weight of active substance is mixed with the specified quantities of solvent and emulsifier and the concentrate diluted with water to the desired concentration to prepare a suitable active substance preparation.

To test the protective efficiency, young plants are sprayed with the active substance preparation in the specified amount. After drying the spray coating, two small pieces of agar grown with Botrytis cinerea are placed on each leaf. The inoculated plants are then placed in a darkened chamber at about 20° C. and 100% relative humidity.

The size of the diseased spots on the leaves is assessed two days after the inoculation. In this situation 0% means an efficiency corresponding to that of the control whereas an efficiency of 100% means that no attack is observed.

In this test the substances according to the invention given in Examples 2, 13, 15, and 16 show an efficiency higher than 90% when used in a quantity of 500 g/ha.

### Example D

Sphaerotheca test (cucumber)/protective

| | |
|---|---|
| Solvent: | 49 parts by weight of N,N-dimethylformamide |
| Emulsifier: | 1 part by weight of alkyl-aryl-polyglycolether |

One part by weight of active substance is mixed with the specified quantities of solvent and emulsifier and the concentrate diluted with water to the desired concentration to prepare a suitable active substance preparation.

To test the protective efficiency, young cucumber plants are sprayed with the active substance preparation in the specified amount. One day after the treatment, the plants are inoculated with a spore suspension of Sphaerotheca fuliginea. The plants are then placed in a glasshouse at 70% relative humidity and a temperature of 23° C.

The assessment is made 7 days after the inoculation. In this situation 0% means an efficiency corresponding to that of the control whereas an efficiency of 100% means that no attack is observed.

In this test the substances according to the invention given in Examples 2 and 8 show an efficiency higher than 90% when used in a quantity of 750 g/ha.

### Example E

Erysiphe test (wheat)/protective

| | |
|---|---|
| Solvent: | 25 parts by weight of N,N-dimethylformamide |
| Emulsifier: | 0.6 parts by weight of alkyl-aryl-polyglycolether |

One part by weight of active substance is mixed with the specified quantities of solvent and emulsifier and the concentrate diluted with water to the desired concentration to prepare a suitable active substance preparation.

To test the protective efficiency, young plants are sprayed with the active substance preparation in the specified amount.

After drying the spray coating, the plants are dusted with spores of Erysiphe graminis f sp. tritici.

The plants are then placed in a glasshouse at a temperature of about 20° C. and a relative humidity of about 80% to promote the development of mildew pustules.

The assessment is made 7 days after the inoculation. In this situation 0% means an efficiency corresponding to that of the control whereas an efficiency of 100% means that no attack is observed.

In this test the substance according to the invention given in Example 2 shows an efficiency higher than 90% when used in a quantity of 500 g/ha.

1. Triazolopyrimidines of the formula 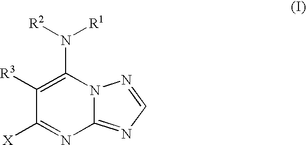
in which
R¹ represents optionally substituted alkyl, optionally substituted alkenyl, optionally substituted alkynyl, optionally substituted cyloalkyl or optionally substituted heterocyclyl,
R² represents a hydrogen or alkyl or
R¹ and R² together with the nitrogen atom to which they are bound, represent an optionally substituted heterocyclic ring,
R³ represents optionally substituted pyridyl or optionally substituted pyrimidyl and
X represents halogen.

2. The triazolopyrimidines of the formula (I) according to claim 1, in which
R¹ represents alkyl with 1 to 6 carbon atoms which can be homogeneously or heterogeneously substituted between one and five times, by halogen, cyano, hydroxy, alkoxy with 1 to 4 carbon atoms and/or cycloalkyl with 3 to 6 carbon atoms, or
R¹ represents alkenyl with 2 to 6 carbon atoms which can be homogeneously or heterogeneously substituted between one and three times, by halogen, cyano, hydroxy, alkoxy with 1 to 4 carbon atoms and/or cycloalkyl with 3 to 6 carbon atoms, or
R¹ represents alkynyl with 3 to 6 carbon atoms which can be homogeneously or heterogeneously substituted between one and three times, by halogen, cyano, hydroxy, alkoxy with 1 to 4 carbon atoms and/or cycloalkyl with 3 to 6 carbon atoms, or
R¹ represents cycloalkyl with 1 to 6 carbon atoms which can be homogeneously or heterogeneously substituted between one and three times, by halogen and/or alkyl with 1 to 4 carbon atoms, or
R¹ represents saturated or unsaturated heterocyclyl with 5 or 6 ring members and 1 to 3 heteroatoms such as nitrogen, oxygen and/or sulphur, wherein the heterocyclyl can be substituted once or twice by halogen, alkyl with 1 to 4 carbon atoms, cyano and/or cycloalkyl with 3 to 6 carbon atoms,
R² represents hydrogen or alkyl with 1 to 4 carbon atoms or
R¹ and R² together with the nitrogen atoms to which they are bound, represent a saturated or unsaturated heterocyclic ring with 3 to 6 ring members, wherein the heterocyclic ring can contain a further nitrogen, oxygen or sulphur atom as ring member and wherein the heterocyclic ring can be optionally substituted up to three times by fluorine, chlorine, bromine, alkyl with 1 to 4 carbon atoms and/or haloalkyl with 1 to 4 carbon atoms and 1 to 9 fluorine and/or chlorine atoms,
R³ represents pyridyl which can be homogenously or heterogeneously substituted between one and four times by
fluorine, chlorine, bromine, cyano,
alkyl, alkoxy, hydroximinoalkyl or alkoximinoalkyl with respectively 1 to 3 carbon atoms,
haloalkyl or haloalkoxy with respectively 1 to 3 carbon atoms
or
R³ represents pyrimidyl which can be homogeneously or heterogeneously substituted between one and three times by
fluorine, chlorine, bromine, cyano,
alkyl, alkoxy, hydroximinoalkyl or alkoximinoalkyl with respectively 1 to 3 carbon atoms,
haloalkyl or haloalkoxy with respectively 1 to 3 carbon atoms and 1 to 7 halogen atoms
and
X represents fluorine, chlorine or bromine.

3. The triazolopyrimidines of formula (I), according to claim 1, in which
R¹ represents a residue of the formula 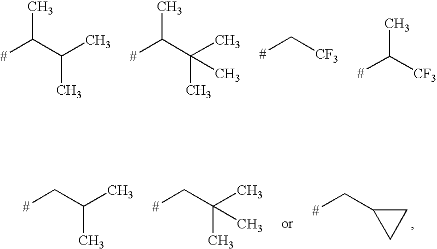
wherein # marks the linking point, or
R¹ represents allyl, dichlorallyl, propargyl, cyclopropyl, cyclopentyl, cyclohexyl, piperidinyl or morpholinyl,
R² represents hydrogen, methyl or ethyl, or
R¹ and R² together with the nitrogen atom to which they are bound, represent pyrrolidinyl, piperidinyl, morpholinyl, thiomorpholinyl, piperazinyl, 3,6-dihydro-1(2H)-piperidinyl or tetrahydro-1(2H)-pyridazinyl, wherein these residues can be substituted by 1 to 3 fluorine atoms, 1 to 3 methyl groups and/or trifluoromethyl,
R³ represents pyridyl which is linked in the 2- or 4-position and can be homogeneously or heterogeneously substituted between one and four times by fluorine, chlorine, bromine, cyano, methyl, ethyl, methoxy, methylthio, hydroximinomethyl, hydroximinoethyl, methoximinomethyl, methoximinoethyl and/or trifluoromethyl, or
R³ represents pyrimidyl which is linked in the 4-position and can be homogeneously or heterogeneously substituted between one and three times by fluorine, chlorine, bromine, cyano, methyl, ethyl, methoxy, methylthio, hydroximinomethyl, hydroximinoethyl, methoximinomethyl, methoximinoethyl and/or trifluoromethyl,
and
X represents fluorine or chlorine.

4. A process for preparing triazolopyrimidines of formula (I) according to any one of claims 1, 2 or 3, characterised in that
(a) dihalogentriazolopyrimidines of the formula 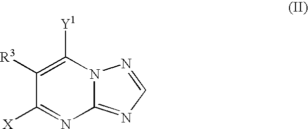
in which
R³ and X have the meanings specified above and
Y¹ represents halogen, are reacted with amines of the formula 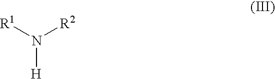
in which
R¹ and R² have the meanings specified above, optionally in the presence of a diluent, optionally in the presence of an acid acceptor and optionally in the presence of a catalyst.

5. A composition for combating undesirable micro-organisms, characterised in that it contains at least one triazolopyrimidine of formula (I) according to claim 1 in addition to extenders and/or surfactants.

6. (canceled)

7. A method for combating undesirable micro-organisms, characterised in that triazolopyrimidines of formula (1) according to claim 1 are applied to the undesirable micro-organisms and/or their habitat.

8. A process for preparing means for combating undesirable micro-organisms, characterised in that triazolopyrimidines of formula (I) according to claim 1 are mixed with extenders and/or surfactants.

9. Dihalogen-triazolopyrimidines of the formula 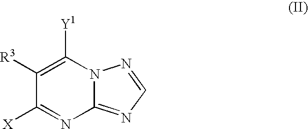
in which
R³ represents optionally substituted pyridyl or optionally substituted pyrimidyl
X represents halogen and
Y¹ represents halogen.

10. A process for preparing dihalogen-triazolopyrimidines of formula (II) according to claim 9, characterised in that
(b) dihydroxy-triazolo-pyrimidines of the formula 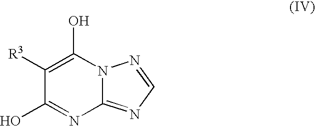
in which
R³ which has the meaning specified in claim 9 is reacted with halogenating agents, optionally in the presence of a diluent.

11. Dihydroxy-triazolo-pyrimidines of the formula 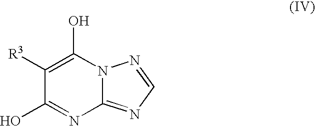
in which
R³ represents optionally substituted pyridyl or optionally substituted pyrimidyl.

12. A process for preparing dihydroxy-triazolo-pyrimidines of the formula (IV) according to claim 11, characterised in that
(c) heteroaryl malonic esters of the formula 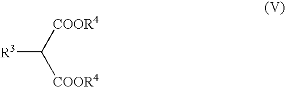
in which
R³ has the meaning specified in claim 11 and
R⁴ represents alkyl with 1 to 4 carbon atoms, is reacted with aminotriazole of the formula 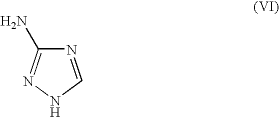
optionally in the presence of a diluent and optionally in the presence of an acid binder.

13. A pyridyl malonic ester of the formula 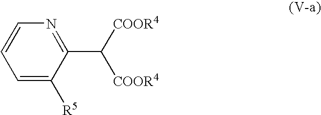
in which
R⁴ represents alkyl with 1 to 4 carbon atoms and
R⁵ represents halogen or haloalkyl.

14. A process for preparing pyridyl malonic esters of the formula (V-a) according to claim 13, characterised in that
(d) pyridine halides of the formula 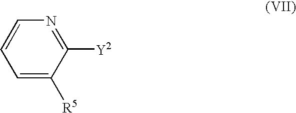
in which
R⁵ has the meaning specified in claim 13 and
Y² represents halogen, are reacted with malonic esters of the formula 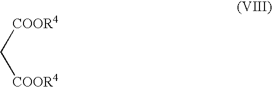
in which
R⁴ has the meaning specified in claim 13, optionally in the presence of a diluent, optionally in the presence of a copper salt and optionally in the presence of an acid acceptor.

15. A pyrimidyl malonic ester of the formula 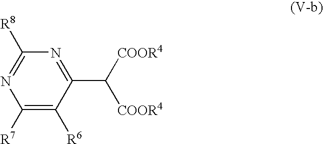
in which
R⁴ represents alkyl with 1 to 4 carbon atoms,
R⁶ represents halogen or haloalkyl and
R⁷ and R⁸ independently of one another represent hydrogen, fluorine, chlorine, bromine, methyl, ethyl or methoxy.

16. A process for preparing pyrimidyl malonic esters of the formula (V-b) according to claim 15, characterised in that
(e) pyrimidine halides of the formula 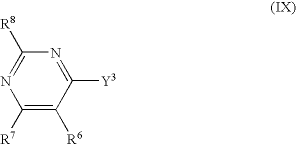
in which
R⁶, R⁷ and R⁸ have the meanings specified in claim 15 and
Y³ represents halogen, are reacted with malonic esters of the formula 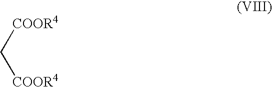
in which
R⁴ has the meaning specified in claim 15, optionally in the presence of a diluent, optionally in the presence of a copper salt and optionally in the presence of an acid acceptor.
